(19) United States
(12) Patent Application Publication
(10) Pub. No.: US 2016/0239619 A1
(43) Pub. Date: Aug. 18, 2016

(54) A UNIQUE METHODOLOGY COMBINING USER ROLES AND CONTEXT AWARE ALGORITHMS FOR PRESENTING CLINICAL INFORMATION, AUDIO, VIDEO AND COMMUNICATION CONTROLS TO SAFELY CAPTURE CAREGIVER ATTENTION, REDUCE INFORMATION OVERLOAD, AND OPTIMIZE WORKFLOW AND DECISION SUPPORT

(71) Applicant: KONINKLIJKE PHILIPS N.V., Eindhoven (NL)

(72) Inventors: Youssef ABOU-HAWILI, ANDOVER, MA (US); John Francis HUMPHRYS, NEEDHAM, MA (US); Larry NIELSEN, BURLINGTON, MA (US); Brian David GROSS, NORTH ANDOVER, MA (US); Kimberly Anne HOFFMAN, SALEM, NH (US)

(21) Appl. No.: 15/025,662

(22) PCT Filed: Oct. 7, 2014
(86) PCT No.: PCT/EP2014/071471

§ 371 Date: Mar. 30, 2016

Related U.S. Application Data
(60) Provisional application No. 61/890,425, filed on Oct. 14, 2013.

(51) Int. Cl. G06F 19/00; G06F 3/0481

(52) U.S. Cl. CPC G06F 19/345; G06F 3/0481

(57) ABSTRACT

A system for generating a role-based user interface includes a patient information database which stores patient data relating to a plurality of patients being treated by one or more caregivers. A caregiver information system stores caregiver data relating to the role, status, and location of the one or more caregivers. A decision support system evaluates the patient data and caregiver data and generates a role-based user interface displaying the most clinically meaningful information to the one or more caregivers based on the evaluation of the patient data and caregiver data.

The present application relates generally to combining user roles and context aware algorithms for presenting clinical information to facilitate workflow optimization. It finds particular application in conjunction with mapping clinical care giver, care giver role, care giver level of experience, caregiver and patient temporal, geo-spatial and situational context, clinical, disease context, sensor inputs, patient and caregiver actions and intentions, and the large corpus of clinical information to assist in collaboration and clinical decision making and will be described with particular reference thereto. However, it is to be understood that it also finds application in other usage scenarios and is not necessarily limited to the aforementioned application.

Typically, clinical care is based on information shared across multiple care roles, including physicians, nurses, patients, family members, and the like, to coordinate care. In many settings, an overload of care information exists that may not be known to all of the members of the team, may not be relevant at a given decision point, or is relevant and is not known to a decision member at a time of a decision.

Add to this the fact that the care team is mobile and unable to physically look at data represented in the EMR for the patient of interest, at the decision time. This creates an environment where errors are made due to incomplete knowledge of existing data, and workflow is impeded as the user needs to “drill” into the vast EMR to get the data, and timely collaboration with the rest of the care team delays good decision making and timely and correct care plan.

To address these problems, the systems and methods of the present application create actionable informational views that are relevant to specific care roles and level of experience, previous user behavior and activities, as well as clinical and patient context. Context can be temporal, geo-spatial, behavioral, clinical, and technological such as the delivery system or device the user is using or wearing. This systems and method of the present application further facilitate care team membership and communication so that decisions, care planning workflow, and coordination/collaboration are improved.

For example, an emergency care physician who is being consulted about a trauma patient that has arrived in the emergency department (ED) is provided an actionable informational view including information such as the patient's clinical history, drug allergies, and interactions which is different from the actionable informational view provided when he/she arrives to the ED and is attending to that patient. In another example, audible information about a trauma patient in the ED is provided to the caregiver when he/she is driving alone in their car, while the caregiver is provided audible information via an ear piece or smart glass if the information does not compromise patient confidentiality if that same physician is driving with other people in the car. Likewise, if the caregiver is in the passenger seat or is stationary or is in an environment or situation that safely allows visual distraction, visual information may be provided in conjunction to audio. Similarly, if a patient is asleep, and the caregiver is in proximity to the patient, the audio system of the caregiver worn or mobile carried delivery device is not activated and is replaced with textual or pictorial information.

Another example of the present application is the automated ability to re-route a patient event to another clinician with the appropriate role who is available and in close proximity to the patient. For example, consider a cardiac patient who is experiencing a critical event and the primary caregiver is either busy, unavailable, or not is the vicinity. This information is continually tracked and kept up-to-date by the system. The system algorithms identify other clinicians with acceptable roles and then determine which of those clinicians is nearest to the patient's location. Once that calculation is completed, the patient event is re-routed to that clinician. Similarly, if a patient in their room requested assistance through a nurse call system or other communication means, when the caregiver enters that room, the nurse call system is automatically silenced and acknowledged to prevent further call repeats and escalations.

The present application provides new and improved methods and system which overcome the above-referenced problems and others.

In accordance with one aspect, a system for generating a role-based user interface is provided. The system includes a patient information database which stores patient data relating to a plurality of patients being treated by one or more caregivers. A caregiver information system stores caregiver data relating to the role and status of the one or more caregivers. A decision support system evaluates the patient data, caregiver data, and context relevant knowledge and generates a role-based user interface displaying the most clinically meaningful and relevant information.

In accordance with another aspect, a system for generating a role based user interface is provided. The system including one or more processors programmed to receive patient data relating to a plurality of patients being treated by one or more caregivers, receive caregiver data relating to the role, status, and location of the one or more caregivers, evaluate the patient data and caregiver situation, and generate a role-based user interface displaying the most clinically meaningful information to the one or more caregivers based on the evaluation of the patient data and caregiver data.

In accordance with another aspect, a method for generating a role based user interface is provided. The method includes receiving patient data relating to a plurality of patients being treated by one or more caregivers, receiving caregiver data relating to the role status, and location of the one or more caregivers, evaluating the patient data and caregiver data, and generating a role-based user interface displaying the most clinically meaningful information to the one or more caregivers based on the evaluation of the patient data and caregiver data.

One advantage resides in reducing alarm fatigue and clinical information overload.

Another advantage resides in improved caregiver collaboration and clinical decision making.

Another advantage resides in improved clinical workflow.

Another advantage resides in improved patient care.

Still further advantages of the present invention will be appreciated to those of ordinary skill in the art upon reading and understanding the following detailed description.

The invention may take form in various components and arrangements of components, and in various steps and arrangement of steps. The drawings are only for purposes of illustrating the preferred embodiments and are not to be construed as limiting the invention.

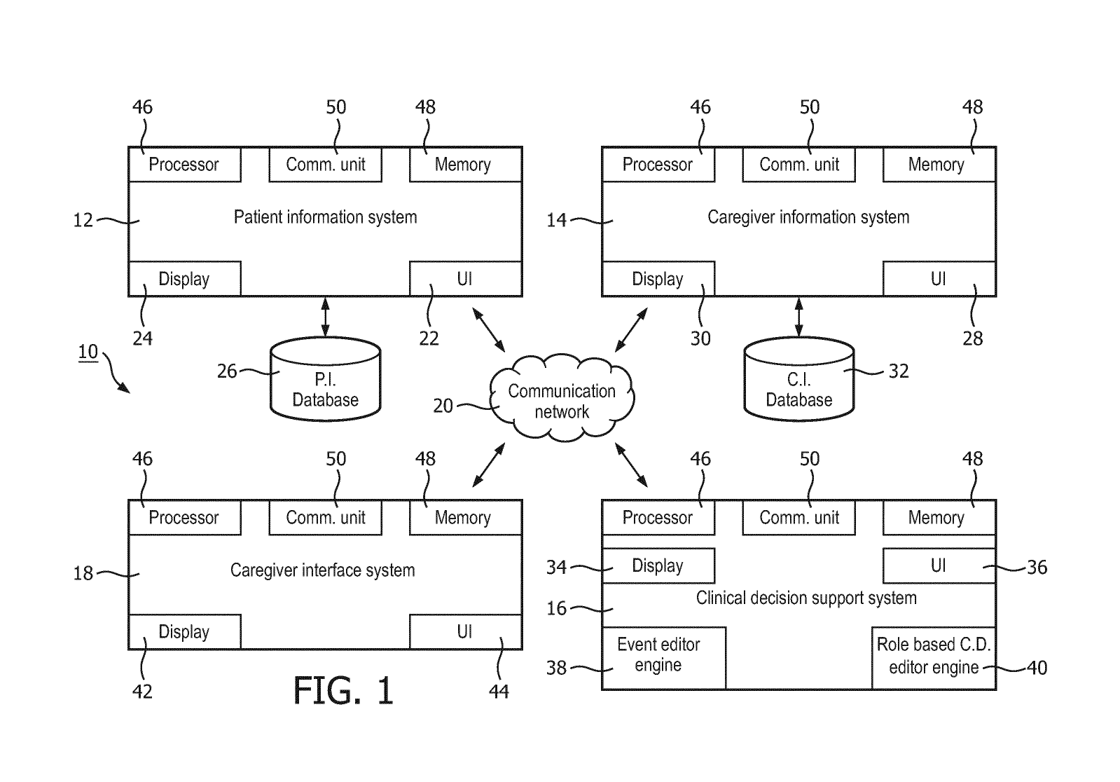

FIG. 1 illustrates a block diagram of a patient monitoring device according to aspects of the present application.

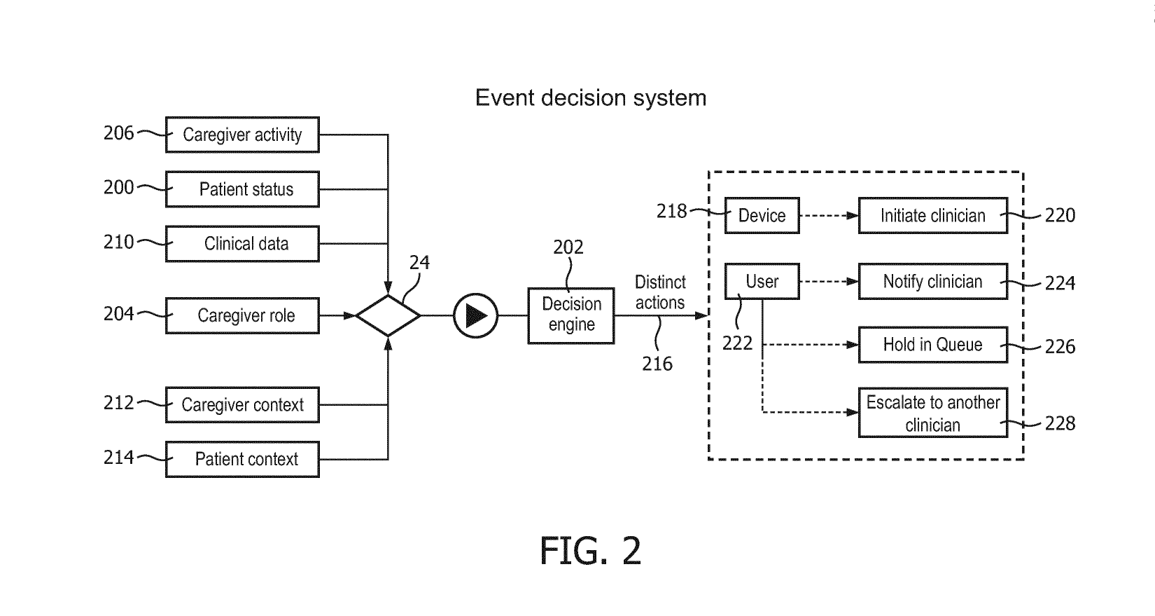

FIG. 2 illustrates a block diagram of the functionality of the event decision system according to aspects of the present application.

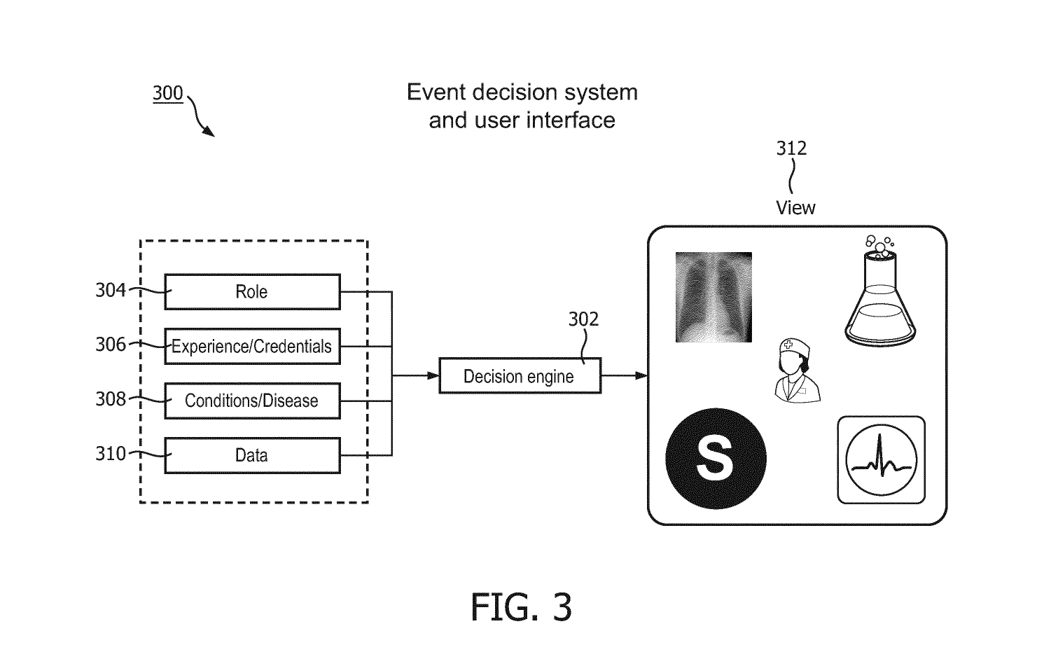

FIG. 3 illustrates a block diagram of the functionality of a role based decision engine according to aspects of the present application.

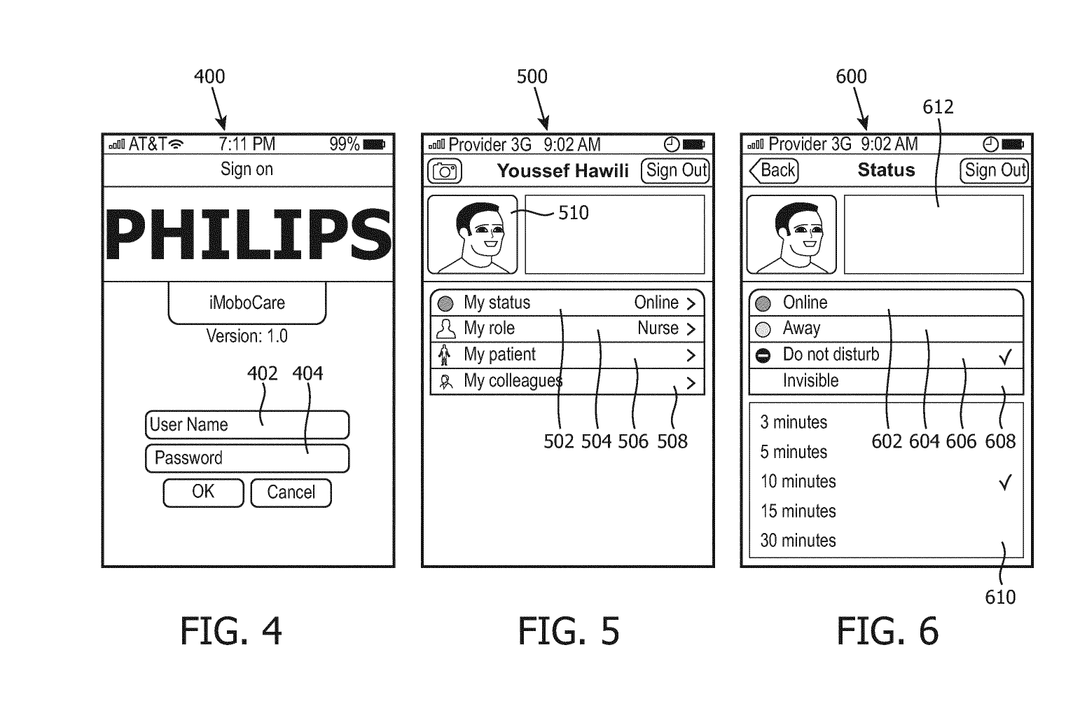

FIG. 4 illustrates an exemplary embodiment of a user authentication interface according to aspects of the present application.

FIG. 5 illustrates an exemplary embodiment of a user presence interface according to aspects of the present application.

FIG. 6 illustrates an exemplary embodiment of a user status interface according to aspects of the present application.

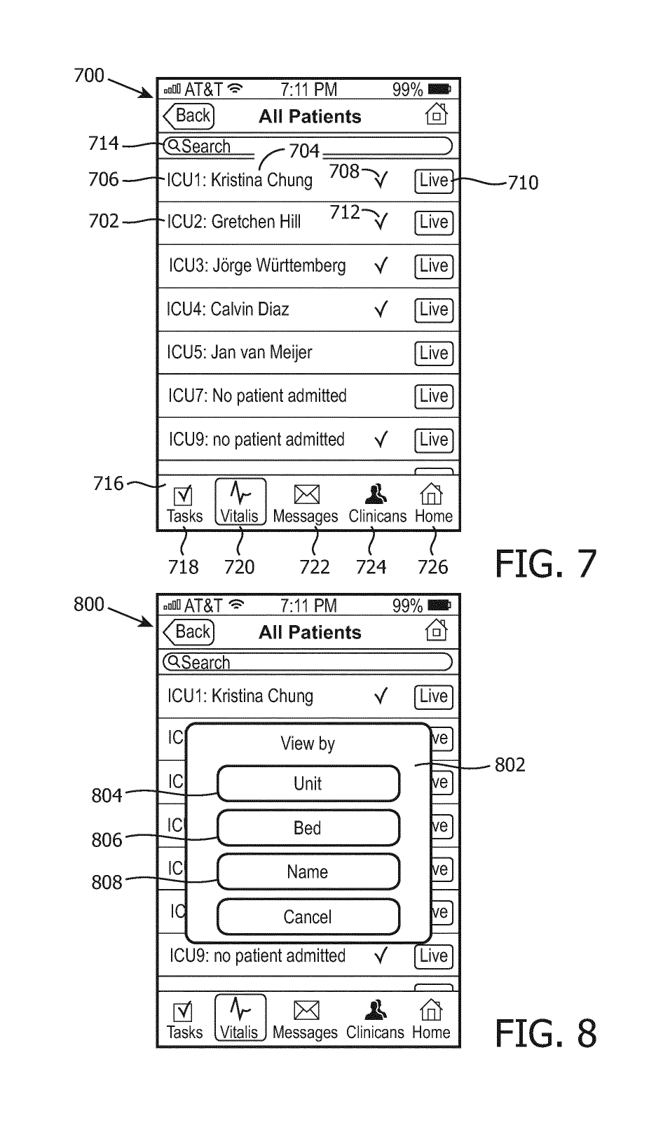

FIG. 7 illustrates an exemplary embodiment of a patient interface according to aspects of the present application.

FIG. 8 illustrates an exemplary embodiment of another patient interface according to aspects of the present application.

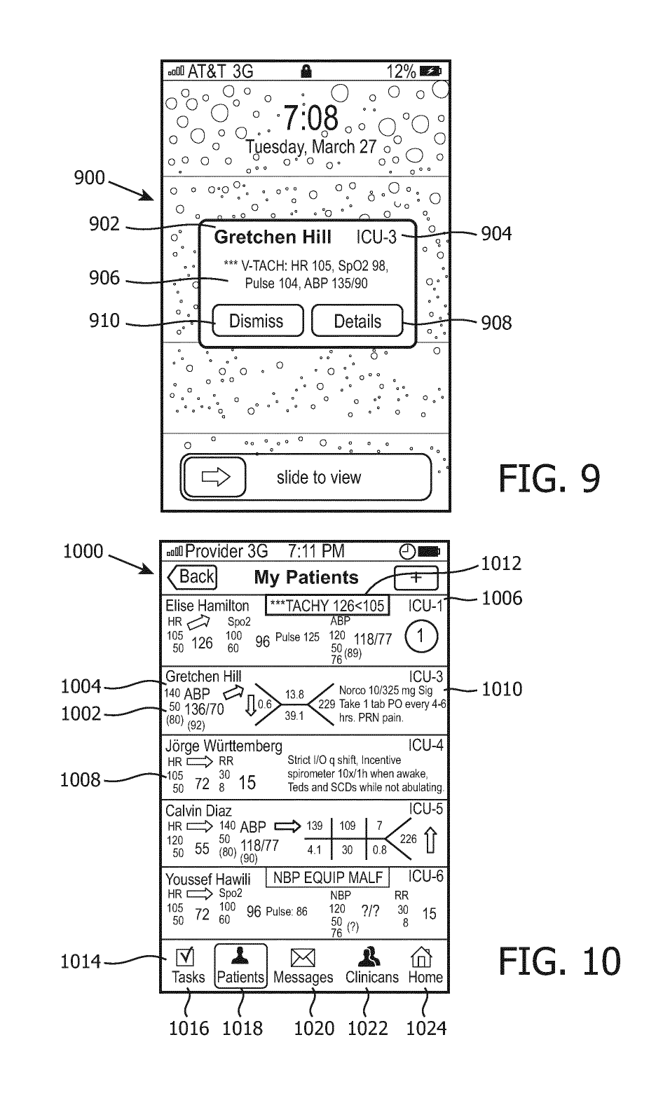

FIG. 9 illustrates an exemplary embodiment of an event notification interface according to aspects of the present application.

FIG. 10 illustrates an exemplary embodiment of a multi-patient interface according to aspects of the present application.

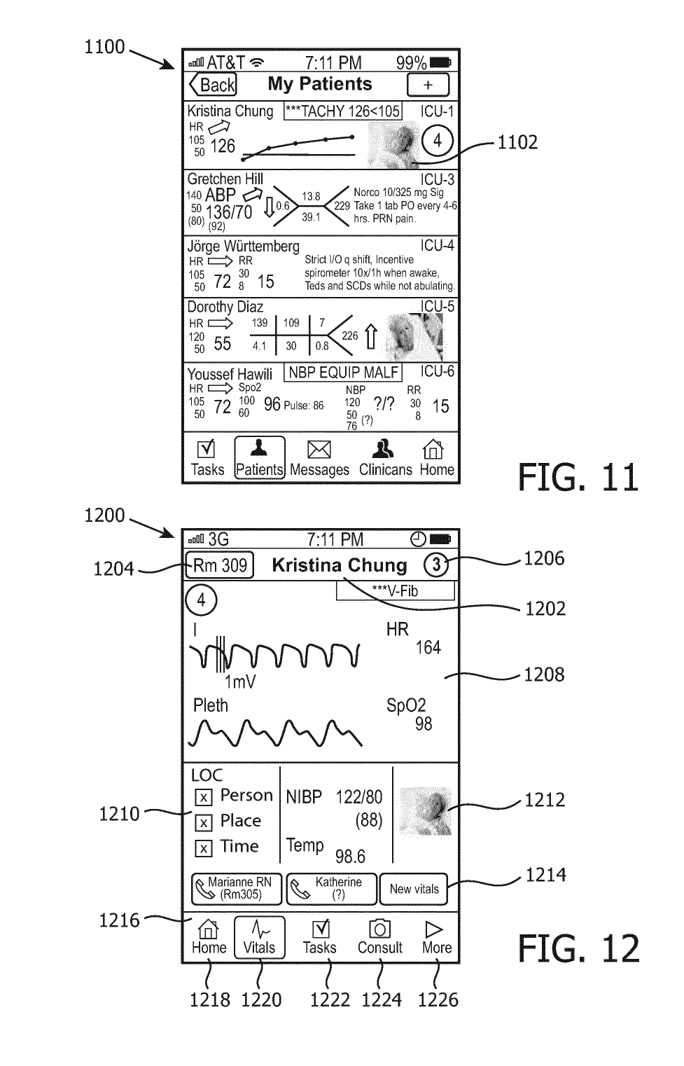

FIG. 11 illustrates an exemplary embodiment of another multi-patient interface according to aspects of the present application.

FIG. 12 illustrates an exemplary embodiment of a single patient interface according to aspects of the present application.

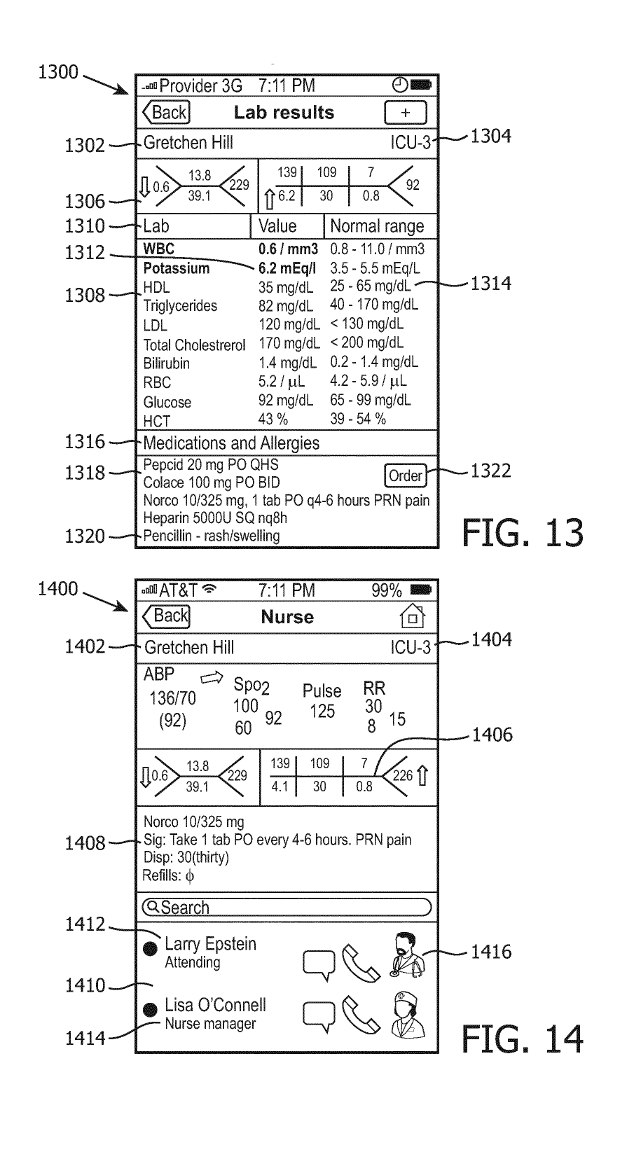

FIG. 13 illustrates an exemplary embodiment of a laboratory results interface according to aspects of the present application.

FIG. 14 illustrates an exemplary embodiment of a care team interface according to aspects of the present application.

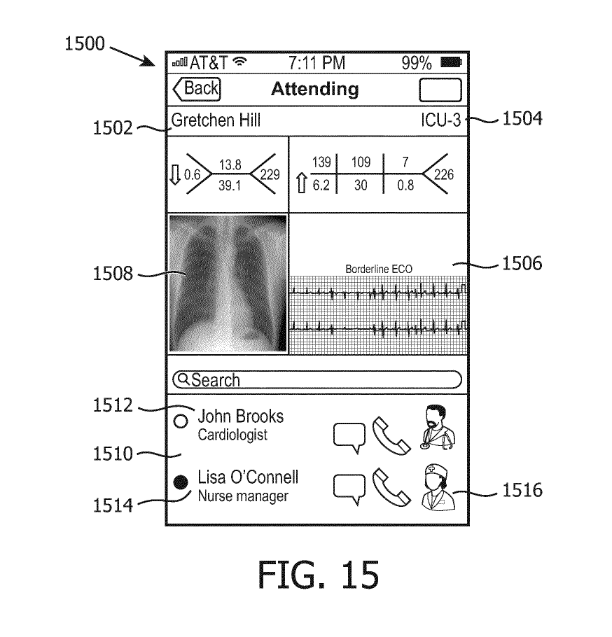

FIG. 15 illustrates an exemplary embodiment of another care team interface according to aspects of the present application.

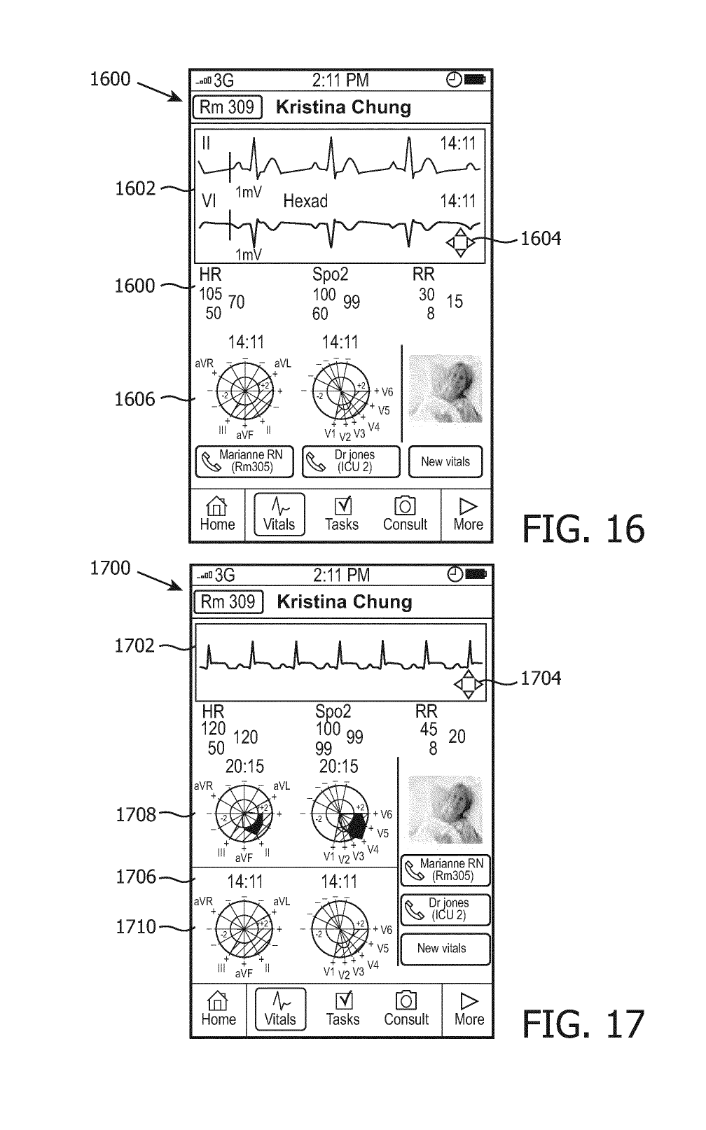

FIG. 16 illustrates an exemplary embodiment of another single patient interface according to aspects of the present application.

FIG. 17 illustrates an exemplary embodiment of another single patient interface according to aspects of the present application.

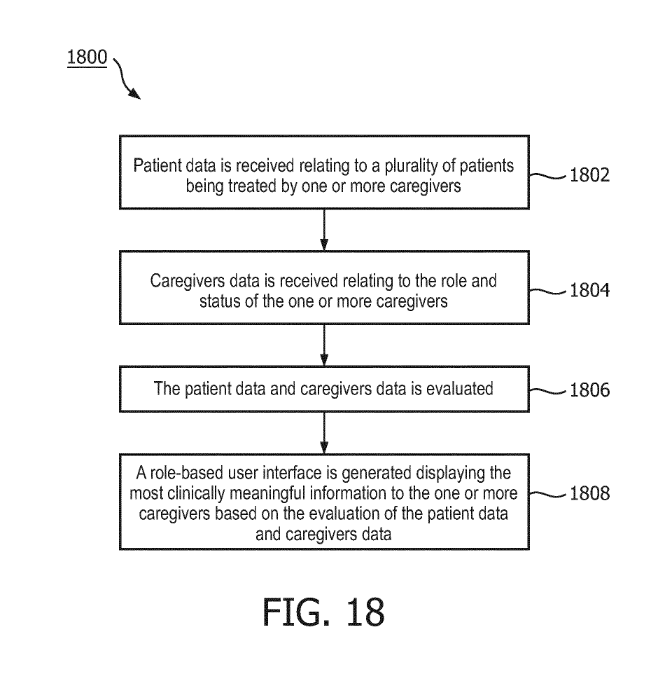

FIG. 18 illustrates a flowchart diagram of a method for generating a role based user interface according to aspects of the present application.

The present application is directed to a systems and methods for mapping clinical care giver, care giver role, care giver level of experience, temporal and location context, patient context, disease context, and the large corpus of clinical information to a mobile application to assist in collaboration and clinical decision making. The major focus of the present application is to provide a unique methodology using context aware algorithms for presenting clinical information, audio, video and communications controls to facilitate workflow optimization and reduce alarm fatigue and information overload. Specifically, clinically relevant events trigger clinically meaningful views to the appropriate users based on user credentials. More specifically, a decision engine is kept advised regarding the status of a patient, the activity of caregivers with responsibility for the patient, patient data, the location of the patient, and the location of the caregivers and the role of each caregiver (doctor, nurse, attendant, etc.) Based on this information, the decision engine notifies the most appropriate caregiver(s) with the most clinically meaningful information. Which clinical data is clinically meaningful is selected based on the role of the caregiver and the other information discussed above. The systems and methods can be used in any environment including hospital, home, and during patient transport in between.

With reference to FIG. 1, a block diagram illustrates one embodiment of an IT infrastructure 10 of a medical institution, such as a hospital. The IT infrastructure 10 suitably includes a patient information system 12, a caregiver information system 14, a clinical decision support system (DSS) 16, caregiver interface system 18, and the like, interconnected via a communications network 20. It is contemplated that the communications network 20 includes one or more of the Internet, Intranet, a local area network, a wide area network, a wireless network, a wired network, a cellular network, a data bus, and the like. It should also be appreciated that the components of the IT infrastructure be located at a central location or at multiple remote locations.

The patient information system 12 stores patient data related to one or more patients being treated by the one or more caregivers of the medical institution. The patient data include physiological data collected from one or more sensors, physiological trending information, laboratory data, imaging data acquired by one or more imaging devices, clinical decision outputs such as early warning scores, and the like. The patient data may also include the patient's medical records, the patient's administrative data (patient's name and location), the patient's medical records, the patient's clinical problem(s), the patient's demographics such as weight, age, family history, co-morbidities, and the like. Further, the patient data can be generated automatically and/or manually. As to the latter, user input devices 22 can be employed. In some embodiments, the patient information system 12 include display devices 24 providing users a user interface within which to manually enter the patient data and/or for displaying generated patient data. In one embodiment, the patient data is stored in the patient information database 26. Examples of patient information systems include, but are not limited to, electronic medical record systems, departmental systems, and the like.

Similarly, the caregiver information system 14 stores caregiver data relating to the roles and status of one or more caregivers. For example, the caregiver information system 14 stores data relating to one or more caregivers including physicians, nurses, patient's family, and the like that are associated with one or more patients. The caregiver data include caregiver roles, clinical experience, credentials (licensure), assigned patients, assigned care units, care domain or setting, caregiver activities, caregiver location, user preferences, and the like. Further, the caregiver data can be generated automatically and/or manually. As to the latter, user input devices 28 can be employed. In some embodiments, the caregiver information systems 14 include display devices 30 providing users a user interface within which to manually enter the caregiver data and/or for displaying generated caregiver data. In one embodiment, the caregiver data are stored in a caregiver database 32. Examples of caregiver information systems include, but are not limited to, clinical administrative databases, caregiver assignment and information databases, and the like.

The DSS 16 stores clinical models and algorithms embodying the clinical support tools or patient decisions aids. The clinical models and algorithms typically include one or more suggested or entered diagnosis and/or treatment options/orders as a function of the patient data and the clinical problem of the patient being treated. Further, the clinical models and algorithms typically generate clinical data that include clinical activities for the various diagnosis and/or treatment options and the clinical context based on the state of the patient and the patient data. Specifically, the clinical models and/or guidelines are determined from the diagnoses and/or treatment orders for patients with specific diseases or conditions and are based on the best available evidence, i.e., based on clinical evidence acquired through scientific method and studies, such as randomized clinical trials. After receiving patient data, the DSS 16 applies the clinical model and algorithm pertinent to the clinical problem of the patient being treated. The DSS 16 then utilizes new clinical data and/or activities to update displays suggesting new treatment options. It should also be contemplated that as more patient data becomes available, the DSS 16 updates the diagnosis and/or treatment options available to the patient. Specifically, the DSS 16 acquires patient data, clinical models and algorithms, and the like and generates clinical data including the clinical context of the patient, the state of care process of the patient, and/or any clinical actions based on the various diagnosis and/or treatment options. The DSS 16 includes a display 34 such as a CRT display, a liquid crystal display, a light emitting diode display, to display the clinical models and algorithms and a user input device 36 such as a keyboard and a mouse, for the clinician to input and/or modify the clinical models and algorithms.

The DSS 16 also includes an event editor engine 38 which maps caregiver data along with patient data, and clinical data to an event that can be targeted to a particular caregiver interface system 18 or user. Specifically, the event editor engine 38 includes a decision engine that algorithmically generates appropriate clinical actions based on patient data, caregiver data, and clinical data. The clinical actions including controlling one or more medical devices such as making measurement more frequently or changing the alarm threshold, generating one or more event notification that notify the caregiver of a critical clinical event such as abnormal vital signs, and the like. If the decision is to notify a caregiver, it determines the action based on the caregiver's role and activity. For example, if the caregiver is an RN and they are currently administering medications, the decision may be to hold the notification for 10 minutes. Alternatively, the decision may be to escalate the request/notification to the caregiver's backup. In this case, a determination is made to see if the backup's role is identical or if it is different. Then the backup and associated data is provided to the engine once again so it can determine the appropriate action. For example, the caregiver's role (RN, MD, NP, etc.) is provided to the engine along with the caregiver's current activity to determine whether the caregiver could perform a certain clinical activity including dispensing medications, patient teaching, performing sterile wound care, etc. Additionally, the caregiver location and patient data are provided to the engine to determine if a close caregiver is available for more immediate care. Caregiver location is specified because actions may be different if the patient is home versus in a medical facility.

The DSS 16 also includes a role based clinical decision editor engine 40 which maps the patient data and clinical data along with the caregiver data to generate user interface views which provide caregiver(s) with the most clinically meaningful information. Specifically, the role based clinical decision editor engine 40 generates user interface views including the patient data and clinical data based on the role of the user. For example, the role based clinical decision editor engine 40 limits the display of patient and/or clinical data based on the role of the caregiver. The role based clinical decision editor engine 40 maps the patient data and clinical data based on determined clinical actions, task engagement of the caregiver, location of the caregiver, experience level of the caregiver, care settings, clinical context, and the like. For example, the role based clinical decision editor engine 40, based on the caregiver role, generates a user interface view that includes a list of all patients assigned to the caregiver, the highest priority physiological data for those assigned patients based on clinical status of the patient, event notification including one or more clinical actions determined by the DSS 16, physiological views based on the patient data and clinical data, care team views which display patient assigned caretaker information, and the like.

The caregiver interface system 18 receives the role-based user interface view from the role based clinical decision editor engine 40 and displays the view to the caregiver on a display 42. The caregiver interface system 18 also includes a user input device 44 such as a touch screen or keyboard and a mouse, for the clinician to input and/or modify the user interface views. Examples of caregiver interface system include, but are not limited to, personal data assistant (PDA), cellular smartphones, personal computers, or the like.

The components of the IT infrastructure 10 suitably include processors 46 executing computer executable instructions embodying the foregoing functionality, where the computer executable instructions are stored on memories 48 associated with the processors 46. It is, however, contemplated that at least some of the foregoing functionality can be implemented in hardware without the use of processors. For example, analog circuitry can be employed. Further, the components of the IT infrastructure 10 include communication units 50 providing the processors 46 an interface from which to communicate over the communications network 20. Even more, although the foregoing components of the IT infrastructure 10 were discretely described, it is to be appreciated that the components can be combined.

With reference to FIG. 2, a block diagram 200 of the functionality of the event decision system is illustrated. As described above, the event editor engine maps caregiver role, activity, location along with patient's clinical data, and status to an event that can be targeted to a device or user. Specifically, the event decision system 200 receives data relating to the role of the caregiver 204, caregiver activity 206, the patient status 208, clinical data 210, patient location 212, and caregiver location 214. Based on the received data, the event decision engine 202 generates distinct actions 216 which are targeted to a device or user. If the distinct actions 216 are targeted to a device 218, the device initiates those distinct actions 220. If the distinct actions 216 are targeted to a user 222, the event decision system will notify the clinician 224, hold in a queue 226, and/or escalate to another clinician 228 based on the status of the caregiver.

With reference to FIG. 3, a block diagram 300 of the functionality of the role-based clinical decision engine is illustrated. As described above, the role-based clinical decision engine 302 maps clinical and disease context along with caregiver role and clinical credentials and experience to prioritized elements in a user interface view. Specifically, the role-based clinical decision engine 302 receives data relating to the role of a caretaker 304, the experience/credentials of the caretaker 306, the conditions/disease of the patient 308, and patient data associated with the patient 310. Based on the received data, the role-based clinical decision engine generates and prioritizes the elements of the user interface view 312 presented to the caretaker.

With reference to FIG. 4, an exemplary embodiment of a user authentication interface 400 generated by decision support system is illustrated. The user authentication interface 400 enables the user to input their credentials including a username 402 and password 404 which the system utilizes to present the mapped patient and clinical data associated with the user. In other embodiments, additional authentication interfaces such as barcode, electronic badges, fingerprint, retinal scans or similar biometric identification systems could be implemented. In one embodiment, the user authentication interface is utilized for authentication of a user and their clinical role. In another embodiment, the user authentication interface is utilized by the user to establish their identification and/or clinical role. In all cases, user authentication is required to view and control any patient identifiable data.

With reference to FIG. 5, an exemplary embodiment of a user presence interface 500 generated by decision support system is illustrated. The user presence interface 500 enables the user to input a personal status 502, input a clinical role 504, view assigned/subscribed patients 506, view subscribed colleagues 508, and the like. The personal status 502 enables the user to indicate whether other users of the caregiver interface system view the user as online, away, busy, idle, invisible, and the like. The clinical role input 504 enables the user to set their clinical role to roles which are previously approved for that user. For example, the clinical role input 504 could enable medical doctor to view the display for the roles of a medical doctor, a nurse practitioner to view the display for a nurse practitioner and a registered nurse but not vice versa. The assigned/subscribed patients view 506 enables the user to view the patient and clinical data associated with the patients which are assigned/subscribed to the user. The subscribed colleagues view 508 enables the user to view the status of subscribed colleagues, view the subscribed colleagues clinical roles, and communicate with subscribed colleagues via message, voice chat, video chat, and the like. The user presence interface 500 also enables the user to insert a picture 510 which is associated with the user's profile.

With reference to FIG. 6, an exemplary embodiment of a user status interface 600 generated by decision support system is illustrated. The user status interface 600 enables the user to indicate their personal status which will be viewed by other users. The user status interface 600 enables the user to select an online status 602, an away status 604, a busy or do not disturb status 606, an offline or invisible status 608, and the like. If the user selects the busy or do not disturb status 606, the user status interface 600 enables the user to selected an approximate time duration 610 for which the user will be busy if desired. The user status interface 500 also enables the user to input messages relating to their status in a status message box 612. It should also be contemplated that the personal status be set manually or automatically. For example, if the user does not access the caregiver interface system for a predetermined amount of time, the DSS system will indicate the user as away.

With reference to FIG. 7, an exemplary embodiment of a patient interface 700 generated by decision support system is illustrated. The patient interface 700 enables the user to select which patient the user would like to subscribe to as well as view patient and clinical data relating to each patient. The patient interface 700 includes a list of patients 702 that are associated with a healthcare facility. The list of patients 702 includes the patients' names 704, the patients' unit bed number 706, subscription icons 708, and an icon 710 for live data viewing. If a user would like to subscribe to a patient on the list 702, the user would select the subscribe icon 710 which would indicate the subscription via a checkmark 712. Likewise, if the user would like to view live data associated with a patient, the user would select the live data viewing icon 710. At the top of the patient interface 700, a search bar 714 is included to enable the user to search for specific patients. The bottom of the patient interface 700 includes navigation icons 716 which enable to navigate between the user's tasks 718, vital signs of subscribed patients 720, messages 722, subscribed clinicians 724, and caregiver interface system interface home 726. These navigation icons 716 are common on most displays.

With reference to FIG. 8, an exemplary embodiment of another patient interface 800 generated by decision support system is illustrated. Patient interface 800 includes the same information as the patient interface 700 illustrated in FIG. 7. However, patient interface 800 enables the user to select how the list of patients is to be viewed via a view by selection window 802. The view by selection window 802 enables the user to view the patients by unit 804, bed 806, patient name 808, and the like.

With reference to FIG. 9, an exemplary embodiment of an event notification interface 900 generated by decision support system is illustrated. The event notification interface 900 enables the user to view a subscribed patient's critical patient and/or clinical data based on the user's role. The event notification interface 900 indicates the patient's name 902, the unit bed number of the patient 904, and any critical patient and/or clinical data 906 associated with the patient. For example, if a subscribed patient has patient data which exceeded a predetermined threshold and is deemed in critical condition, the DSS system will display an event notification indicate the user of the event. The event notification interface 900 can also display information regarding scheduled clinical activities associated to a patient. It should also be appreciated that the event notification interface 900 can also display the events of one or more patients at a time. The event notification interface 900 also enables the user to view details 908 regarding the event. If the user wishes to view details regarding the event, the DSS system limits the displayed patient and/or clinical data based on the role of the user. The event notification interface 900 also enables the user to dismiss 910 the event.

With reference to FIG. 10, an exemplary embodiment of a multi-patient interface 1000 generated by decision support system is illustrated. The multi-patient interface 1000 enables the user to view patient and/or clinical data associated with multiple patients in a single display. The multi-patient interface 1000 includes a list of patient 1002 and their associated patient and/or clinical data. For each patient, the multi-patient interface 1000 displays the name of the patient 1004; the patient's unit bed number 1006; the patient data 1008 associated with the patient including physiological data, early warning scores, trending information, laboratory results, and the like; clinical data 1010 including scheduled clinical activities. In one embodiment, the DSS system displays the highest priority patient and/or clinical data associated with a patient based on the patient's status, clinical problem, and abnormal lab results. In another embodiment, the user selects which patient and/or clinical data is displayed for each patient manually. It should also be appreciated that the DSS limits the displayed patient and/or clinical data of the multi-patient interface 1000 based on the role of the user. The multi-patient interface 1000 also displays event notification icons 1012 based on a patient's critical patient and/or clinical data. The bottom of the multi-patient interface 1000 includes the common navigation icons 1014 which enables the user to navigate between the user's tasks 1016, vital signs of subscribed patients 1018, messages 1020, subscribed clinicians 1022, and caregiver interface system interface home 1024.

With reference to FIG. 11, an exemplary embodiment of another multi-patient interface 1100 generated by decision support system is illustrated. Multi-patient interface 1100 includes the same information as the multi-patient interface 1000 illustrated in FIG. 10. However, multi-patient interface 1100 includes a photograph or video chat window 1102 of the patient being monitored. The video chat window 1102 enables the user to communicate with the patient being displayed when selected. It is also contemplated that the video chat window 1102 enables the user to message, voice chat, and the like with the client if the video chat feed is unavailable. It should also be appreciated that the video chat window 1102 be used for monitoring purposes with the ability of the patient to communicate with the user being disabled.

With reference to FIG. 12, an exemplary embodiment of a single patient interface 1200 generated by decision support system is illustrated. The single patient interface 1200 enables the user to view patient and/or clinical data associated with a single patient in a single display. The single patient interface 1200 includes the patient's name 1202, their location 1204, and an early warning score 1206 at the top of the display. The single patient interface 1200 also displays patient data 1208 associated with the patient including physiological data, early warning scores, trending information, laboratory results, and the like and/or clinical data 1210 including scheduled clinical activities associated with the patient. In one embodiment, the DSS system displays the highest priority patient and/or clinical data associated with a patient based on the patient's status and clinical problem. In another embodiment, the user selects which patient and/or clinical data is displayed for each patient manually. It should also be appreciated that the DSS limits the displayed patient and/or clinical data of the single patient interface 1200 based on the role of the user. A video chat window 1212 enables the user to communicate with the patient being displayed when selected. It is also contemplated that the video chat window 1212 enable the user to message, voice chat, and the like with the client if the video chat feed is unavailable. It should also be appreciated that the video chat window 1212 be used for monitoring purposes with the ability of the patient to communicate with the user being disabled. The single patient interface 1200 also enables the user to communicate with colleagues that are also assigned to the patient via one or more communication icons 1214. The bottom of the single patient interface 1200 includes the common navigation icons 1216 which enables the user to navigate between the caregiver interface system interface home 1218, the vital signs of subscribed patients 1220, the user's tasks 1222, subscribed clinicians 1222, a consultation interface 1224, and the like. The More icon 1226, provides scanning to additional buttons if necessary.

With reference to FIG. 13, an exemplary embodiment of a laboratory results interface 1300 generated by decision support system is illustrated. The laboratory results interface 1300 enables the user to view patient and/or clinical data, particularly laboratory results, associated with a single patient in a single display. The laboratory results interface 1300 includes the patient's name 1302 and unit bed number 1304 at the top of the display. The laboratory results interface 1300 displays the laboratory data in a graphical fishbone presentation 1306. The laboratory results interface 1300 also includes a laboratory results window 1308 which includes a list of all laboratory tests 1310, the results of those tests 1312, and the normal range of the results 1314. Abnormal lab results are highlighted with a graphical directional icon indicating whether a value is below a normal lower limit and/or is trending downward or above a normal upper limit and/or is trending upwards. In one embodiment, the DSS system displays the highest priority patient and/or clinical data associated with a patient based on the patient's status and clinical problem. In another embodiment, the user selects which patient and/or clinical data is displayed for each patient manually. It should also be appreciated that the DSS system limits the displayed patient and/or clinical data of the laboratory results interface 1300 based on the role of the user. The laboratory results interface 1300 also includes a medication and allergies window 1316 which displays the prescribed medications 1318 and allergies 1320 of the patient and enables a qualified user, in this case the attending physician, to re-order medication with an order icon 1322.

With reference to FIG. 14, an exemplary embodiment of a care team interface 1400 generated by decision support system is illustrated. The care team interface 1400 enables the user, in this case a nurse, to view patient and/or clinical data associated with a single patient based on the user's role. The care team interface 1400 includes the patient's name 1402 and unit bed number 1404 at the top of the display. The care team interface 1400 also displays patient data 1406 associated with the patient including physiological data, early warning scores, trending information, graphical display of laboratory results, and the like and/or clinical data 1408 including scheduled clinical activities and orders associated with the patient. In one embodiment, the DSS system displays the highest priority patient and/or clinical data associated with a patient based on the patient's status and clinical problem. In another embodiment, the user selects which patient and/or clinical data is displayed for each patient manually. It should also be appreciated that the DSS limits the displayed patient and/or clinical data of the care team interface 1400 based on the role of the user. The care team interface 1400 also includes a list of relevant personal 1410 to the patient including an attending physician 1412 and nurse manager 1414. The list of relevant personal 1410 also include communication icons 1416 to establish communication with the attending physician and nurse manager via messaging, voice chat, video chat, and the like.

With reference to FIG. 15, an exemplary embodiment of another care team interface 1500 generated by decision support system is illustrated. The care team interface 1500 enables the user, in this case an attending physician, to view patient and/or clinical data associated with a single patient based on the user's role. The care team interface 1500 includes the patient's name 1502 and unit 1504 at the top of the display. The care team interface 1500 also displays patient data 1506 associated with the patient including physiological data, early warning scores, trending information, laboratory results, medical images, 12-lead ECGs, and the like and/or clinical data including scheduled clinical activities associated with the patient. In one embodiment, the DSS system displays the highest priority patient and/or clinical data associated with a patient based on the patient's status and clinical problem. In another embodiment, the user selects which patient and/or clinical data is displayed for each patient manually. It should also be appreciated that the DSS system limits the displayed patient and/or clinical data of the care team interface 1500 based on the role of the user. The care team interface 1500 also includes a list of relevant personal 1510 to the patient including a cardiologist 1512 and nurse manager 1514. The list of relevant personal 1510 also includes communication icons 1516 to establish communication with the cardiologist and nurse manager via messaging, voice chat, video chat, and the like.

FIGS. 14 and 15 demonstrate a significant concept of the present application. Specifically, a large amount of patient and clinical data is available for most clinical situations. This information has some value to all caregivers, but limited value to others based on the clinical situation and context. Tailoring the information provided to caregivers based on their role and the situation may improve care and outcomes.

For example, consider a patient with pneumonia. For the nurse attending to the patient, vital signs and lab values as shown in FIG. 14 are appropriate. In general, nurses are not skilled in reading X-Rays, so providing an X-Ray image (as shown in FIG. 15) to the nurse has little or no value. However, that information is important to provide to the attending physician. Providing the X-Ray and an ECG strip to the physician adds value. Physicians are skilled in reading both X-Rays and ECGs, and most likely would request that information. Realizing that physicians will want this information and proactively providing it in a timely manner improves workflow and patient care.

FIGS. 16 and 17 take this concept a step further. Consider a patient who begins complaining of chest pain. The physician could be notified with a sample ECG (notice arrows indicating that you can scroll backwards and forwards through time as well as up and down to view the other leads) and two ST Map images (showing all leads). The physician reviews this information and determines that the patient is stable from a cardiac point of view. Approximately six hours later the patient again complains of chest pain. At this time, the physician can be notified with additional and relevant information. Providing this information in a timely manner to the clinician with the appropriate role allows care to be provided with minimal delays.

With reference to FIG. 16, an exemplary embodiment of another single patient interface 1600 generated by decision support system is illustrated. Patient interface 1600 includes the same information as patient interface 1200 illustrated in FIG. 12. However, patient interface 1600 includes a sample ECG 1602 (notice arrows 1604 indicating that you can scroll backwards and forwards through time as well as up and down to view the other leads) and two ST Map images 1606 (showing all leads).

With reference to FIG. 17, an exemplary embodiment of another single patient interface 1700 generated by decision support system is illustrated. Patient interface 1700 includes the same information as patient interface 1600 illustrated in FIG. 16. However, patient interface 1700 includes a sample ECG 1702 (notice arrows 1704 indicating that you can scroll backwards and forwards through time as well as up and down to view the other leads) and four ST Map images 1706 (showing all leads) including current ST Map images 1708 and historic ST Map images 1710.

With reference to FIG. 18, a flowchart diagram 1800 of a method for generating a role based user interface is illustrated. Although each of the blocks in the diagram is described sequentially in a logical order, it is not to be assumed that the system processes the described information in any particular order or arrangement. In a step 1802, patient data is received relating to a plurality of patients being treated by one or more caregivers. In a step 1804, caregiver data is received relating to the role and status of the one or more caregivers. In a step 1806, the patient data and caregiver data is evaluated. In a step 1808, a role-based user interface is generated displaying the most clinically meaningful information to the one or more caregivers based on the evaluation of the patient data and caregiver data.

As used herein, a memory includes one or more of a non-transient computer readable medium; a magnetic disk or other magnetic storage medium; an optical disk or other optical storage medium; a random access memory (RAM), read-only memory (ROM), or other electronic memory device or chip or set of operatively interconnected chips; an Internet/Intranet server from which the stored instructions may be retrieved via the Internet/Intranet or a local area network; or so forth. Further, as used herein, a processor includes one or more of a microprocessor, a microcontroller, a graphic processing unit (GPU), an application-specific integrated circuit (ASIC), a field-programmable gate array (FPGA), personal data assistant (PDA), cellular smartphones, mobile watches, computing glass, and similar body worn, implanted or carried mobile gear; a user input device includes one or more of a mouse, a keyboard, a touch screen display, one or more buttons, one or more switches, one or more toggles, and the like; and a display device includes one or more of a LCD display, an LED display, a plasma display, a projection display, a touch screen display, and the like.

The invention has been described with reference to the preferred embodiments. Modifications and alterations may occur to others upon reading and understanding the preceding detailed description. It is intended that the invention be constructed as including all such modifications and alterations insofar as they come within the scope of the appended claims or the equivalents thereof.

1. A system for generating a role-based user interface, said system comprising:
a patient information database which stores patient data relating to a plurality of patients being treated by one or more caregivers;
a caregiver information system which stores caregiver data relating to the role and status of the one or more caregivers; and
a decision support system which evaluates the patient data, caregiver data, and context relevant knowledge and generates a role-based user interface displaying the most clinically meaningful and relevant information to the one or more caregivers.

2. (canceled)

3. (canceled)

4. (canceled)

5. (canceled)

6. (canceled)

7. (canceled)

8. (canceled)

9. (canceled)

10. A system according to claim 1, wherein the decision support system includes:
one or more processors programmed to:
receive the patient data relating to a plurality of patients being treated by one or more caregivers;
receive the caregiver data relating to the role, status, and location of the one or more caregivers;
evaluate the patient data and caregiver data; and
generate a role-based user interface displaying the most clinically meaningful information to the one or more caregivers based on the evaluation of the patient data and caregiver data.

11. The system according to claim 10, wherein the one or more processors are further programmed to:
map the patient data with the caregiver data to prioritize elements displayed in the user interface.

12. The system according to claim 10, wherein the one or more processors are further programmed to:
limit the patient data displayed on the user interface based on the caregiver data.

13. The system according to claim 10, wherein the one or more processors are further programmed to:
evaluate the patient data and generates clinical data indicative of one or more diagnosis and/or treatment options.

14. The system according to claim 13, wherein the one or more processors are further programmed to:
map the patient data and clinical data to generate clinical events which can be targeted to one or more users or one or more medical devices.

15. The system according to claim 14, wherein the one or more processors are further programmed to:
target and display the clinical events to one or more caregivers based on the caregiver data.

16. The system according to claim 15, wherein the one or more processors are further programmed to:
manage the clinical events based on caregiver data and at least one of holds the clinical event or redistributes the clinical event when a caregiver should not be interrupted.

17. A method for generating a role based user interface, said method comprising:
receiving patient data relating to a plurality of patients being treated by one or more caregivers;
receiving caregiver data relating to the role, status, and location of the one or more caregivers;
evaluating the patient data and caregiver data; and
generating a role-based user interface displaying the most clinically meaningful information to the one or more caregivers based on the evaluation of the patient data and caregiver data.

18. The method according to claim 17, further including:
mapping the patient data with the caregiver data to prioritize elements displayed in the user interface.

19. The method according to claim 17, further including:
limiting the patient data displayed on the user interface based on the caregiver data.

20. The method according to claim 17, further including:
evaluating the patient data and generates clinical data indicative of one or more diagnosis and/or treatment options.

21. The method to claim 20, further including:
mapping the patient data and clinical data to generate clinical events which can be targeted to one or more users or one or more medical devices.
